(19) United States
(12) Patent Application Publication
(10) Pub. No.: US 2026/0183218 A1
(43) Pub. Date: Jul. 2, 2026

(54) MASCARA COMPOSITION

(71) Applicant: Coty Inc., New York, NY (US)

(72) Inventors: Davian David-Ramphal, New York, NY (US); Tamar Lesnoy, New York, NY (US); Stella Macharia, New York, NY (US); Scarlet Romeno, New York, NY (US); Marissa Solomon, New York, NY (US); Jakyung Yoon, New York, NY (US); Alice Kim, New York, NY (US)

(21) Appl. No.: 19/127,345

(22) PCT Filed: Nov. 16, 2023
(86) PCT No.: PCT/US2023/080119

§ 371 Date: May 5, 2025

Related U.S. Application Data
(60) Provisional application No. 63/426,435, filed on Nov. 18, 2022.

(51) Int. Cl. A61K 8/81; A61K 8/06; A61K 8/36; A61K 8/37; A61K 8/60; A61K 8/92; A61Q 1/10

(52) U.S. Cl. CPC A61K 8/8182; A61K 8/062; A61K 8/361; A61K 8/375; A61K 8/60; A61K 8/8152; A61K 8/922; A61Q 1/10

(57) ABSTRACT

Discussed herein is a mascara formulation and related methods. A formulation may include an emulsifier. A formulation may include a water phase comprising a thickener including a 0.3% to 1.0% Tara gum sufficiently dispersed in the water phase, wherein the water phase comprises a viscosity of at least 25,000 CPS on a Brookfield measurement. A formulation may include an oil phase comprising a wax system being about 5% to about 25% of the formulation, the wax system including a first wax having a melting point below 50° C. and a second wax having a melting point above 60° C.

### CROSS-REFERENCE TO RELATED APPLICATIONS

This application claims priority to U.S. Application Ser. No. 63/426,435, filed Nov. 18, 2022, entitled “MASCARA COMPOSITION,” which is hereby incorporated by reference in their entirety.

### BACKGROUND

Mascara formulations have been used for many years to increase the thickness, length and overall aesthetic appearance of human eyelashes.

### SUMMARY OF THE INVENTION

Various aspects relate to a mascara formulation. In some aspects, the techniques described herein relate to a cosmetic formulation for mascara, the formulation including: an emulsifier; a water phase including a thickener including a 0.3% to 1.0% Tara gum sufficiently dispersed in the water phase, wherein the water phase includes a viscosity of at least 25,000 CPS on a Brookfield measurement; and an oil phase including a wax system being about 5% to about 25% of the formulation, the wax system including a first wax having a melting point below 50° C. and a second wax having a melting point above 60° C.

In some aspects, the mascara formulation is an oil in water emulsion. The emulsion includes a water phase. The water phase includes about 0.3 wt % to about 0.7 wt % of sufficiently dispersed Tara gum (CAS No. 39300-88-4) to yield a water phase exhibiting a Brookfield viscosity of at least 0.50 CPS. The emulsion includes an oil phase. The oil phase includes an emulsifier selected from emulsifiers classified as natural origin cosmetic ingredients (NOI) having in a range of from about 0.1 to about 1 as determined by ISO 16128-1:2016 and the emulsifier does not comprise triethanolamine. The oil phase also includes a wax system in a range of from about 5 wt % to about 25 wt % of the composition. When applied, the mascara formulation exhibits sufficient volume and sufficient definition as assessed by Example 1, Example 2, or both.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 depicts a chart of data from the Examples discussed herein.

FIG. 2 depicts a chart of data from the Examples discussed herein.

FIG. 3A depicts an example mascara brush for use with the formulations discussed herein.

FIG. 3B depicts an example mascara brush for use with the formulations discussed herein.

FIG. 3C depicts a chart of data from the Examples discussed herein.

### DETAILED DESCRIPTION OF THE INVENTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order without departing from the principles of the invention, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%.

The term “substantially free of” as used herein can mean having none or having a trivial amount of, such that the amount of material present does not affect the material properties of the composition including the material, such that about 0 wt % to about 5 wt % of the composition is the material, or about 0 wt % to about 1 wt %, or about 5 wt % or less, or less than or equal to about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

### Mascara Formulation

According to various aspects of the present disclosure, a mascara formulation can deliver volume, wear, definition, and removal benefits. The mascara formulation can have a good volume. The mascara formulations can have good tack, viscosity, wax components, and polymer selection which contribute to the beneficial volume.

The mascara formulation can be considered to be a vegan formulation. As understood herein a “vegan formulation” refers to a mascara formulation that includes component(s) that are not derived from an animal product and to the extent possible not tested on an animal. In some examples 100 wt % of the components of the mascara formulation are vegan.

Discussed herein, the mascara formulation is an oil in water emulsion with both a water phase and an oil phase, in addition to an emulsifier. The water phase of the formulation can include, for example, thickeners or gellants, film formers, various story ingredients, and a wax system. The oil phase can include, for example, thickeners or gellants, film formers, various story ingredients, waxes, or combinations thereof. These various components of the mascara formulation can include polymers, silicones, waxes, pigments, other components, and combinations thereof. The polymers can provide a flexible non-flaking film that also prevents the mascara formulation from smudging. The silicones can improve water resistance and make the mascara wear longer. The waxes can provide adhesion and have an impact on thickening lashes.

The mascara formulation discussed herein allows for quick deposition of a nice, smooth film. The composition is flake-proof and easy to remove. The composition is defined as instantly volumizing, light weight resulting in a volumized look.

In an example, the composition includes a sufficiently dispersed Tara gum in the water phase, to achieve a desired viscosity. It has a wax system that includes C10-18 triglycerides, which has a low melting point, in combination with other high melting point waxes at a specific ratio. It contains an emulsifier system that includes sucrose stearate in combination with glyceryl stearate. Furthermore, the formulation includes of a film forming system including an acrylate/VP Hexadecene copolymer according to a defined ratio. These unique ingredient combinations starts with incredible deposition that allows for thickening, full bodied volume and softer lashes as it dries out.

An example mascara formulation is summarized below in Table 1.

TABLE 1
Example mascara formulation.

| | |
|---|---|
| Thickener | Tara gum |
| Emulsifier | Sucrose stearate |
| | Glyceryl stearate |
| Wax system | C10-18 triglycerides |
| | Additional high melting point wax(es) |
| Pigments | Various |
| Film forming system | Acrylate |
| | VP Hexadecene copolymer |

Various other components

Thickener or Gellant. The mascara formulation can include one or more thickeners or gellants in the water phase. The thickeners or gellants can aid in the viscosity and tack of the mascara formulation.

The thickeners or gellants can include, for example, Tara gum (CAS No. 39300-88-4). The Tara gum can be sufficiently dispersed in the water phase to allow for a desired viscosity of the overall formulation.

Tara gum can replace many different gums used in traditional mascara formulations. For example, Tara gum can be a direct replacement to xanthan gum. However, a drawback of Tara gum is that it can have a tendency to agglomerate or become lumpy, which can adversely affect the definition of the mascara formulation. However, Tara gum can be used if it is sufficiently dispersed in the mascara formulation.

A visual metric to determine if the Tara gum is sufficiently dispersed is to assess whether the Tara gum has “fisheyes.” Fisheyes are a description given to an agglomeration of dry gums when inadequately dispersed in water. These gums are originally presented in powder form. When introduced to water, the gums begin to hydrate and, in the process, increase the viscosity of water. Good gum incorporation means that the swelling/hydration process is completed after full dispersion of the gum. If there is still some gum that is visible in the vessel during mixing, then that pooling of gum is what is referred to as fish eyes. No amount of additional shear or energy increase will disperse/break up such agglomeration.

The Tara gum is present in a range of from about 0.3 wt % to about 1.0 wt % of the mascara formulation, 0.4 wt % to about 0.9 wt %, less than, equal to, or greater than about 0.3 wt %, 0.35, 0.4, 0.45, 0.5, 0.55, 0.6, 0.65, 0.7, 0.75, 0.8, 0.85, 0.9, 0.95 or about 1.0 wt %.

The total amount of sufficiently dispersed Tara gum can influence the viscosity of the water phase. For example, a viscosity of the water phase, measured with a Brookfield viscosimeter, is at least 25,000 CPS.

The water phase, in which the Tara gum is sufficiently dispersed, can optionally include additional components. The oil phase of the mascara formulation can include an emulsifier and a wax system, among other components.

Emulsifier. The mascara formulation can include an emulsifier to aid in mixing of the water phase and the oil phase. The emulsifier can be micellar, lamellar, or mixed. In the example mascara formulation, the emulsifier system can include sucrose stearate, optionally in combination with glyceryl stearate.

The emulsifier can be present in a range of from about 0.1 wt % to about 15 wt % of the mascara formulation, about 0.1 wt % to about 1 wt %, less than, equal to, or greater than about 0.1 wt %, 0.5, 1, 2, 3, 4, 5, 6, 7, 8, 9, 10, 11, 12, 13, 14, or about 15 wt %.

The instant mascara formulation can be free of triethanolamine xantham, hydroxyethyl cellulose, acacia gum, or a mixture thereof. In the instant disclosure, the emulsifier includes emulsifiers classified as natural origin cosmetic ingredients (NOI) having a minimum value of 1 as determined by ISO 16128-1:2016.

In a particular example, the emulsifier is sucrose stearate in combination with glyceryl stearate. The sucrose stearate and glyceryl stearate can be in a ratio of about 1:1 in the emulsifier system.

Waxes. The mascara formulation can include one or more waxes in the oil phase. The waxes can help provide for removal of the mascara formulation. The waxes can include, for example, a first wax with a lower melting point below about 50° C., and second wax with a higher melting point above about 60° C. The wax system can include a low melting point wax in combination with one or more other high melting point waxes.

The low melting point wax can be an organic wax or a synthetic wax. The low melting point wax can be, for example, C10-18 triglycerides, or may be other waxes or butters, such as shea butter, berry wax, myrica, tribehenin, or other appropriate low melting point waxes. The low melting point wax can have a melting point of about 50° C. or less, of about 40° C. or less, of about 30° C. or less, or a melting point of about 32° C.

The low melting point wax can be in an amount of about 10 wt % to about 50 wt % of the wax system, of about 10 wt %, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or about 50 wt %.

The low melting point wax can have a penetration value of about 0 to 30, of about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or about 30.

The wax system can include one or more additional waxes that have a higher melting point. The higher melting point wax or waxes can be organic or synthetic. Such waxes can include comprises carnauba wax, euphorbia cerifera cera, cera microcristallina, hydrogenated vegetable oil, stearyl stearate, stearic acid, and combinations thereof. The high melting point wax can have a melting point of about 60° C. or more, of about 70° C. or more, of about 80° C. or more, or of about 90° C. or more.

The wax system as a whole is present in a range of from about 5 wt % to about 25 wt % of the mascara formulation, about 10 wt % to about 20 wt %, less than, equal to, or greater than about 5 wt %, 6, 7, 8, 9, 10, 11, 12, 13, 14, 15, 16, 17, 18, 19, or about 20 wt %.

The low melting point wax can have a penetration value of about 0 to 10, of about 0, 1, 2, 3, 4, 5, 6, 7, 8, 9, or about 10.

The wax system can include a plurality of waxes. For example, the wax system can include four waxes. In a four wax mixture, a first wax can have a melting point in a range of from about 66° C. to about 75° C. (68° C. to 73° C., less than, equal to, or greater than about 66° C., 67, 68, 69, 70, 71, 72, 73, 74, or 75° C.), a second wax can have a melting point in a range of from about 46° C. to about 56° C. (48° C. to 54° C., less than, equal to, or greater than about 46° C., 47, 48, 49, 50, 51, 52, 53, or 54° C.), a third wax can have a melting point in a range of from about 78° C. to about 88° C. (80° C. to 86° C., less than, equal to, or greater than about 78° C., 79, 80, 81, 82, 83, 84, 85, 86, 87, or 75° C.), a fourth wax can have a melting point in a range of from about 43° C. to about 57° C. (45° C. to 55° C., less than, equal to, or greater than about 43° C., 44, 45, 46, 47, 48, 49, 50, 51, 52, 53, 54, 55, 56, or 57° C.), or a mixture thereof. A weight ratio of the first wax to second wax to third wax to fourth wax can be in a range of from about 1:1:0.3:0.8 to about 1:1:0.5:0.9, or 1:1:0.4:0.8.

In a specific example, the first wax is carnauba wax, the second wax is myrica wax, the third wax is berry wax, and the fourth wax is candelilla wax. The berry wax can be a wax provided from or derived from a berry product. Berry wax is not present in all versions of the composition. An example of a suitable berry wax is verniciflua pell cera (CAS no. 8001-39-6). In some aspects the wax system can include a synthetic beeswax. A synthetic beeswax refers to a synthetically derived mimic of beeswax. More specifically, the synthetic beeswax is not an animal product or derived from an animal product. As an example, the synthetic beeswax includes a mixture of cera microcristallina (CAS no 63231-60-7), hydrogenated vegetable oil (CAS no. 68334-28-1), stearyl stearate (CAS no. 2778-96-3), and stearic acid (CAS no. 57-11-4). In another example, the synthetic beeswax includes a mixture of ozokerite (CAS no 64742-33-2), hydrogenated vegetable oil (CAS no. 68334-28-1), stearyl stearate (CAS no. 2778-96-3), and stearic acid (CAS no. 57-11-4).

The instantly disclosed mascara formulation can include numerous additional components.

Pigments and colorants. The instantly disclosed mascara formulation can include a colorant or pigment component, which can be present in the mascara formulation in a range of from about 5 wt % to about 20 wt % of the mascara formulation, about 8 wt % to about 12 wt % of the mascara formulation, less than, equal to, or greater than about 5 wt %, 10, 15, or 20 wt % of the mascara formulation. Colorants suitable for use in the present mascara formulations include, but are not limited to, dyes, pigments, lakes, and mixture thereof. (e.g., organic or inorganic pigments and colorants approved for use in eye-area cosmetics by CTFA and/or the FDA.) Exemplary inorganic pigments include particles of iron oxides (e.g., yellow, brown, red, black), titanium dioxides, iron sulfides, ultramarines, chromium oxides (e.g., green) or other conventional pigments used in cosmetic formulations. Examples of organic pigments include D&C Black No. 2, D&C Black No. 3, FD&C Red No. 40, D&C Green No. 5, FD&C Blue No. 1, and FD&C Yellow No. 5. Examples of lake dyes include various acid dyes which are laked with aluminum, calcium or barium. Additional colorants for use herein include annatto, caramel, carmine, β-carotene, bismuth oxychloride, ferric ammonium ferrocyanide, ferric ferrocyanide, chromium hydroxides (e.g., green), guanine, mica, aluminum powder, bronze powder, copper powder, manganese violet, zinc oxide. Suitable colorants along with their chemical structure are described in, e.g., 21 C.F.R. Part 74 and in the CTFA Cosmetic Ingredient Handbook, (1988), published by the Cosmetics, Toiletry and Fragrances Association, Inc. Other colorants may also be used as they are developed and determined safe.

Film Formers. The mascara formulation can further include a film former component. In an example, the mascara formulation can include multiple film formers as a film forming system. The film forming system can be in the water phase.

The total film formers can be in a range of from about 0.20 wt % to about 5 wt % of the mascara formulation, about 0.50 wt % to about 2 wt % of the mascara formulation, less than, equal to, or greater than about 0.20 wt %, 0.50, 1, 1.5, 2, 2.5, 3, 3.5, 4, 4.5, or about 5 wt % of the mascara formulation.

In an example, the formulation includes of a film forming system of an acrylates copolymer to VP Hexadecene copolymer according to a ratio of about 10:1 to about 20:1. In an example, the film forming system can be about 1.0 wt % to about 20 wt % of the formulation.

As an example, the film former component can include polyvinylpyrrolidone. Additional suitable film formers include rosin esters, which are derived from rosin. Rosin is a solid form of resin obtained from conifers (e.g., pine tree sap). Rosin is known to include a mixture of closely related rosin acids, especially abietic acid, characterized by three fused six-carbon rings, C═C double bonds that vary in number and location, and a single carboxylic acid group. Commercial methods for obtaining rosin are also known and include, e.g., distilling the volatile turpentine from oleoresin exuded from the wound of living pine trees to obtain gum rosin, or the chemical separation of tall oil, which is a byproduct of the wood pulp industry, to obtain tall oil rosin. The carboxylic acid group of a rosin acid can be converted to an ester by reacting the acid with an alcohol. Esterification of rosin modifies the softening point, adhesiveness, cohesiveness, and melted viscosity of the material. The alcohols typically used to make rosin esters are methanol, tri-ethylene-glycol, glycerol, and pentaerythritol. Tall oil rosin is esterified with glycerol to form tall oil glycerides, a mixture of rosin acids, and esters of glycerol. Tall oil glycerides are available from, for example, Arizona Chemical Co. Glyceryl rosinate, is the ester of a rosin acid reacted with glycerol. Pentaerythrityl rosinate, sometimes referred to as pentaerythritol rosinate, is the ester of a rosin acid reacted with pentaerythritol. Pentaerythritol rosinate is commonly used as a skin conditioning agent-emollient and viscosity increasing agent, and is nonaqueous in cosmetic formulations.

Rosin includes a conjugated system of C═C double bonds, which makes it susceptible to oxidation, isomerization and other chemical reactions. A common method to improve stability is to hydrogenate the rosin, for example, by the addition of hydrogen to the conjugated C═C double bonds in the presence of a catalyst to form saturated ring structures. Hydrogenated rosin esters have specific advantages over non-hydrogenated (e.g., lighter color, improved stability, and reduced skin sensitization). The hydrogenated versions of pentaerythrityl rosinate and glyceryl rosinate (i.e., pentaerythrityl hydrogenated rosinate (“PHR”) and glyceryl hydrogenated rosinate (“GHR”)) are suitable for use herein.

A variety of other story ingredients can be included in the mascara formulation, including but not limited to conditioning agents, fibers, butter, functional ingredients, fragrance, and others.

Carrier elements. The mascara formulation can further include carrier to help deliver the desired mascara components (e.g., pigments and film formers) to the eyelash or eyelid. In certain embodiments, the semi-permanent mascara formulation may include a volatile carrier that quickly volatilizes from the surface of the eyelashes or eyelid, leaving the desired components behind. The volatile carrier may be present at 10% to 85%, 15% to 80%, or even 20% to 70% by weight based on the weight of the composition. Nonlimiting examples of suitable volatile carriers include volatile hydrocarbons, volatile silicones, and mixtures thereof.

Hydrocarbon oils suitable for use as a carrier in the present mascara formulations include those having boiling points in the range of 60-260° C., such as hydrocarbon oils having a carbon chain length of from C8 to C20 (e.g., C8 to C20 isoparaffins). Particularly suitable examples of isoparaffins include those selected from the group consisting of isododecane, isohexadecane, isoeicosane, 2,2,4-trimethylpentane, 2,3-dimethylhexane and mixtures thereof. Isodecane is available from Presperse under the brand name Permethyl 99A and has the formula: CH—₃(CH₂)10CH.

Rheology modifying components. The mascara formulation can further include a rheology modifying component. The rheology modifying component can be in a range of from about 2 wt % to about 15 wt % of the mascara formulation, about 8 wt % to about 12 wt % of the mascara formulation, less than, equal to, or greater than about 2 wt %, 3, 4, 5, 6, 7, 8, 9, 10, 11, 12, 13, 14, or about 15 wt % of the mascara formulation. Examples of suitable rheology modifying components include thickeners. Thickeners for use in the present mascara formulations can be selected from the group consisting of waxes such as carnauba wax, candelilla wax, and polyethylene wax; particles such as disteardimonium hectorite, kaolin, silica, and magnesium carbonate; polymers; viscous hydrocarbons; and combinations thereof. Waxes are selected to maintain the film durability of the mascara formulation.

Active ingredients. The compositions of the present mascara formulations may comprise a safe and effective amount of a biological, chemical, nutraceutical, or pharmaceutical active, or a combination thereof. Biological actives may include prostaglandins, antimicrobials, antibacterials, biocides, preservatives, proteins, amino acids, peptides, hormones, growth factors, enzymes (e.g., glutathione sulphydryl oxidase, transglutaminase), therapeutics, oligonucleotides, genetic materials (e.g., DNA, RNA), and combinations thereof. Chemical actives may include dyes, surfactants, sensates, hair conditioners, hair dyes, hair growth agents, hair removers, hair growth inhibitors, hair styling gels, and combinations thereof. Nutraceutical actives may include proteins, preservatives, vitamins, food-additive materials, and combinations thereof. Pharmaceutical actives may include antibiotics, drugs, hair growth agents, hair removers, hair growth inhibitors, and combinations thereof.

Other additives. The mascara formulation can further include an oil-soluble or dispersible additive which may include fatty alcohols such as Guerbet alcohols based on fatty alcohols having from 6 to 30, preferably from 10 to 20 carbon atoms including lauryl alcohol, cetyl alcohol, stearyl alcohol, cetearyl alcohol, oleyl alcohol, benzoates of C₁₂-C₁₅ alcohols, acetylated lanolin alcohol, etc. Especially suitable is stearyl alcohol. The oil-soluble or dispersible additive may include fatty acids such as Linear fatty acids of C₆-C₂₄, branched C₆-C₁₃carboxylic acids, hydroxycarboxylic acids, caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, elaeostearic acid, arachidic acid, gadoleic acid, behenic acid and erucic acid and technical-grade mixtures thereof (obtained, for example, in the pressure removal of natural fats and oils, in the reduction of aldehydes from Roelen's oxosynthesis or in the dimerization of unsaturated fatty acids). Further components that can be used are dicarboxylic acids of C2-Cu, such as adipic acid, succinic acid, and maleic acid. Aromatic carboxylic acids, saturated and/or unsaturated, especially benzoic acid, can be used. Additional components that can be used as the oil soluble or dispersible additive include carboxylic acid salts; alkaline soaps of sodium, potassium and ammonium; metallic soaps of calcium or magnesium; organic basis soaps such as lauric, palmitic, stearic and oleic acid, etc., alkyl phosphates or phosphoric acid esters: acid phosphate, diethanolamine phosphate, potassium cetyl phosphate

Other useful oil-soluble or dispersible additives comprise mild surfactants, super-fatting agents, consistency regulators, additional thickeners, polymers, stabilizers, biologically active ingredients, deodorizing active ingredients, anti-dandruff agents, film formers, swelling agents, UV light-protective factors, antioxidants, preservatives, insect repellents, solubilizers, colorants, bacteria-inhibiting agents, hair conditioning agents, vitamins, and the like.

Other components. There are any number of further components that can be included in the mascara formulation such as a surfactant component, a skin conditioner component, a humectant component, or another additive component. Other additives can include chamomile and cucumber extract, jojoba oil, camella oil, aloe butter, maracuja oil, cell-u-lash, marula oil, castor oil, ceramide 2, avocado butter, or a mixture thereof.

As compared to other mascara formulations, the instant mascara formulation is substantially free of natural beeswax. Additionally, as compared to other mascara formulations, the instant mascara formulation can be substantially free of sympeptide, procapil, bamboo extract, sodium hyaluronate, collagen, keratin, talc, phenoxyethanol, BPD-500, or a mixture thereof.

### Method of Making Mascara Formulation

The instantly described mascara formulations can be made according to a variety of suitable methods. As an example, water and the ingredients in the water phase (aside from Tara gum) are added to a beaker and exposed to low shear conditions (about 100 rpm) at room temperatures (about 25° C.). Tara gum can be premixed with glycerin and propanediol to create a slurry which is them added to the water phase with the shear increased from about 200 rpm to about 500 rpm. The resultant formulation is mixed with the constituents of the oil phase to form the mascara formulation.

All waxes can be melted (e.g., change from a solid state to a liquid or molten state). Once the waxes are sufficiently melted, other components such as pigments, emulsifiers, and the like are added to create the oil phase. Also, the water phase is prepared by dispersing Tara gum in water. The water phase and the il phase are then combined to form the oil in water emulsion.

The mascara formulation can be contained in an applicator assembly. A variety of conventional applicators are known in the art (e.g., molded plastic applicators and twisted-wire brush applicators). Mascara formulations may act as a paste or semi-solid on the brush, rather than as a flowable liquid. Thus, the primary method of depositing such mascaras onto the mascara brush is by inserting the brush into a supply of mascara, typically contained in a bottle or similar container, so that the mascara is deposited on the head (i.e., bristles and core) of the brush. Excess mascara is removed from the head of the brush, especially the outermost portions of the bristles spaced away from the core, via a wiper when the brush is withdrawn from the mascara container. Ideally, a suitable amount of mascara remains at least on the core of the brush for application to the eyelashes of a user. When the brush is used as intended, the lashes of a user are moved past the bristles of the brush via the spaces between the bristles such that the lashes come into contact with the mascara-containing core and, optionally, portions of the bristles (e.g., the inner portions). Thus, it is the characteristics of the mascara (e.g., coherent strength & rigidity) in combination with the applicator and wiper that provides the primary mechanism of product control and dosing. Because conventional mascaras tend to be thick enough for the mascara to remain on the core of the brush, it is not uncommon for the spacing of the bristles to be determined primarily by the desired lash separation/clustering. But with the desire to provide lower viscosity mascaras, the spacing of the bristles becomes important for providing suitable product retention on the brush as well as the desired amount of lash separation/clustering.

### Application of a Mascara Formulation

A suitable mascara product according to the present disclosure includes a container (e.g., bottle or the like) for storing a supply of the present semi-permanent mascara formulation; an applicator (e.g., twisted-wire brush) for transferring mascara from the container to the eyelashes of a user; and a supply of the present semi-permanent mascara formulation disposed in the container. The applicator may include a handle which is removably and/or re-attachably joined to the container (e.g., with screw threads, snap collar, or the like).

An advantage of the instant mascara formulations is that it is volumizing. That is multiple layers of the mascara formulation can be applied to a keratinous fiber (e.g., an eyelash) to build the volume of the fiber. More specifically, the mascara formulations of the instant disclosure allow for sufficient volume after only two strokes, only five strokes, or only ten strokes to the keratinous fiber. As understood, a stroke applies to a portion of the mascara formulation, that disposed on a brush, being applied in a single motion to the keratinous fiber. Examples of suitable brushes include an elastomeric brush (e.g., a molded brush with elastomeric bristles) a twisted-wire brush, or both. It is possible to build the volume of an eyelash by about 10% to about 50% relative to an eyelash that is free of the mascara formulation, about 15% to about 40%, less than, equal to, or greater than about 10%, 15, 20, 25, 30, 35, 40, 45, or about 50%.

In an example, a twisted brush with hourglass shape and tapered end to fully grab lashes can be used. In an example, a twisted wire brush with tapered shape and grooves between fibers to allow for loading of formulation can be used.

### Examples

### Example 1. Sample Mascara Formulation

An example of a suitable mascara formulation includes the composition of Table 2:

TABLE 2
Example Formulation.

| Ingredient wt % in | |
|---|---|
| mascara formulation | Ingredient Name |
| 35-40 | Water Purified |
| 8-17 | Worlée Micromer C60/42 |
| 4-10 | ALDO MS CG |
| 74-10 | SunPuro Black Iron Oxide C33-7001 |
| 2-6 | Carnauba Wax #1 Flakes NF SP-63 |
| 2-6 | Kahlwax 6279L Myrica |
| 1-5 | Kahlwax 2039L Candelilla Wax |
| 1-5 | Vellaplex MB |
| 1-53 | SISTERNA SUCROSE ESTER SP70-C |
| 0.5-4 | KahlWax 6290 Berry |
| 0.5-3 | Zemea Propanediol |
| 0.3-2.3 | Indopol H-300 |
| 0.3-3 | Emulmetik 100J |
| 0.2-2.5 | Greenolene 6928 |
| 0.1-2.2 | L-Arginine C |
| 0.2-1 | BENZYL ALCOHOL DD |
| 0.2-1 | Glycerine USP |
| 0.2-1 | Aglumix 01 (260901) |
| 0.1-1 | Caprylyl Glycol |
| 0.05-0.50 | DL Panthenol |
| 0.05-0.3 | Silfoam SE2 |
| 0.03-0.2 | EDTA 3NA 2H20 |

In some aspects it is possible to remove the following components: Sucrose Stearate, Vellaplex MB (Capryloyl Glycerin/Sebacic Acid Copolymer), EDTA, Kahlwax 6279L (Myrica Cerifera (Bayberry) Fruit Wax), and Kahlwax 6290 (Rhus Verniciflua Peel Wax). The removed components can be replaced with Sucrose Stearate, Glyceryl Stearate SE, and Water), Unimer U-151 (VP/Hexedecene Copolymer), Natrlquest E30 (Trisodium Ethylenediamine Disuccinate), Lipocire (C10/18 Triglyceride), and Kahlwax 6103 (Cera Microcristallina, Hydrogenated Vegetable Oil, Stearyl Stearate, and Stearic Acid), while maintaining the characteristics described herein.

An example of a second suitable mascara formulation includes the composition of Table 3:

TABLE 3
Example formulation

| Ingredient wt % in | |
|---|---|
| mascara formulation | Ingredient Name |
| 40-45% | Water Purified |
| 10-14% | Worlée Micromer C60/42 |
| 1-4% | ALDO MS CG |
| 5-8% | SunPuro Black Iron Oxide C33-7001 |
| 2-6% | Carnauba Wax #1 Flakes NF SP-63 |
| 2-6% | Kahlwax 2039L Candelilla Wax |
| 0.5-2% | Zemea Propanediol |
| 0.5-2.5% | Indopol H-300 |
| 0.5-2.5% | Emulmetik 100J |
| 0.5-1.5% | Priolene 6928 |
| 0.3-1.5% | L-Arginine C |
| 0.3-1.5% | BENZYL ALCOHOL DD |
| 0.3-1% | Solagum |
| 0.3-1% | Caprylyl Glycol |
| 0.1-0.4% | DL Panthenol |
| 0.1-0.4% | Silfoam SE2 |
| 1-5% | Kahlwax 6103 MB |
| 0.5-3% | Lipocire S AG |
| 0.2-1% | Hystrene 5016 NF Flake Veg (10555) |
| 1-3% | Unimer U-151 |
| 5-9% | Tego Care SE 121 MB |
| 0.05-2% | Natrlquest E30 |

A panelist has their eye lashes inspect to ensure that they are clean, have no cosmetic product applied, and are dry. A scientist takes a high-resolution image of the panelist's bare lashes. Fresh bare images are required to capture any changes to lash topography. Mascara formulations of Table 3 are shared with the panelist. Instructions provided if brush requires redirection. The panelist opens tube, pulls out wand and applies 15 strokes to one eye. The panelist re-dips wand back to bottle, pulls out recharged brush and applies 15 more strokes to the other eye. This is repeated one more time (thus netting two applications or two overall “strokes”). The panelist proceeds back to the camera location. The panelist will then position their face and the scientist proceeds to manipulate the platforms accordingly to get the panelists eye into range for both a forward facing and upward facing image of each eye. Professional evaluators examine the images and determine whether the eyelashes demonstrate sufficient volume. The evaluation is counseled also by consumer feedback. It is expected that the mascara formulation yields sufficient volume. It is also expected that the composition yields sufficient definition. Definition is understood to refer to the separation of lashes with each coating, revealing a full fringe of awakened even spaced lash hair without clumping that enhances an open eye look.

The volumizing properties of the mascara formulation of Table 2 can be quantitively determined by applying the mascara to a panelist eyelash as explained in Example 1 or to a synthetic brush with bristles that mimic a human eyelash. Images are captured of the uncoated eyelash or bristle. Images are also taken of the coated eyelash or bristle. The images are processed using a program that is able to measure the area of the eyelashes or bristles. The program calculates the % growth as equal to ((surface area of loaded brush-surface area of bare brush)/surface area of bare brush)*100. It is expected that at least a 20% growth will be achieved using the mascara formulation. The test is also expected to show sufficient definition in the eyelashes. Definition is understood to refer to the separation of lashes with each coating, revealing a full fringe of awakened even spaced lash hair without clumping that enhances an open eye look.

### Example 2. Example Mascara Formulations

Three example mascara formulations were produced and tested for a variety of properties, including but not limited to viscosity, rheology, volume, Crockmeter testing, and others. The three example mascara formulations are summarized in Table 4 below:

TABLE 4
Sample mascara formulations.

| Sample ID | Thickener | Wax System | Film Formers | Emulsifier |
|---|---|---|---|---|
| Sample A | Tara gum | Lipocire | acrylates | sucrose |
| | | (C10-18 | copolymer | stearate |
| | | triglycerides) | VP hexadecane | glyceryl |
| | | Carnauba wax | copolymer | stearate |
| Sample B | Acacia | Lipocire | acrylates | sucrose |
| | | (C10-18 | copolymer | stearate |
| | | triglycerides) | VP hexadecane | glyceryl |
| | | Carnauba wax | copolymer | stearate |
| Sample C | Tara gum | Shea butter | acrylates | sucrose |
| | | Carnauba wax | copolymer | stearate |
| | | | VP hexadecane | glyceryl |
| | | | copolymer | stearate |

Additional components were the same across all Samples A to C. The first formulation, Sample A, included the sufficiently dispersed Tara gum and the wax system with a first wax having a low melting point and a second wax having a higher melting point.

Viscosity and Rheology testing. The Samples were tested for viscosity and rheology to show wear of the mascara formulation. The Samples were tested with a Brookfield device at room temperature, Tf, at a speed of 0.5 rpm for a time of 1 minute. The Samples were tested for various rheological properties, including yield stress and viscosity with standard procedures. The Samples were tested both for viscosity at 10 seconds (to emulate pick-up and initial deposit of the formulation) and at 100 second (to emulate the experience during application).

Crockmeter testing. The Samples were tested with the Crockmeter, a rub tester used to determine the amount of color transferred from textile materials to other surfaces by rubbing, and to help analyze smudge and smear. The methods used to test the Samples include a the AATC method 116-2013. This testing was applied to examine the Sample mascara formulations on glass slides.

In the Crockmeter testing, the Samples, in addition to blank slides and control samples, were tested under a dry rub approach and a wet rub approach. Each of the samples was tested with the manual crank for 10 complete rotations according to a metronome. For samples with liquid, the liquid was applied immediately before the application of the Crockmeter, and the samples were then allowed to dry overnight. Here, the Samples were tested both under dry conditions and with squalene, which emulated sebum or sweat. The performance of each sample was rated under a standardized Crockmeter score.

Rub test and abrasion testing. The Samples were tested for rubbing and abrasion to show wear of the mascara formulation. For the rub test, the sample were taken at 37° C. and a relative humidity of 60. The number of minute to film failure in these conditions during rubbing were recorded. For the abrasion testing, the number of passes for film failure was recorded.

Results. The results of the sample testing are shown in Table 5 below:

TABLE 5
Sample testing results.

| | Sample A | Sample B | Sample C |
|---|---|---|---|
| | Tara and | Acacia and | Tara and |
| | Lipocire | Lipocire | Shea butter |
| Viscosity | 4,300,000 | 7,160,000 | 6,240,000 |
| Rheology | 993.038 | 180.515 | 610.001 |
| Yield Stress | | | |
| (Pa) | | | |
| Rheology | 27.2 | 25.1 | 54.9 |
| Viscosity | | | |
| at 10/s | | | |
| Rheology | 4.19 | 2.72 | 5 |
| Viscosity | | | |
| at 100/s | | | |
| Crockmeter | 1.98 | 1.7 | 2.19 |
| (dry) | | | |
| Crockmeter | 3.29 | 5.65 | 5.9 |
| (squalene) | | | |
| Rub test | 15++ | 11 | 14 |
| Abrasion | 600 | 300 | 200 |

As shown in Table 5, the inventive Sample A performed surprisingly well compared to the comparative Sample B and Sample C. First, for viscosity and rheology, Samples A to C maintained relatively high viscosity. The Samples A and B with the low melting point wax Lipocire performed well on the dry Crockmeter test. The Samples A and C with Tara gum performed well on the rub test. The Sample A performed very well on the abrasion test.

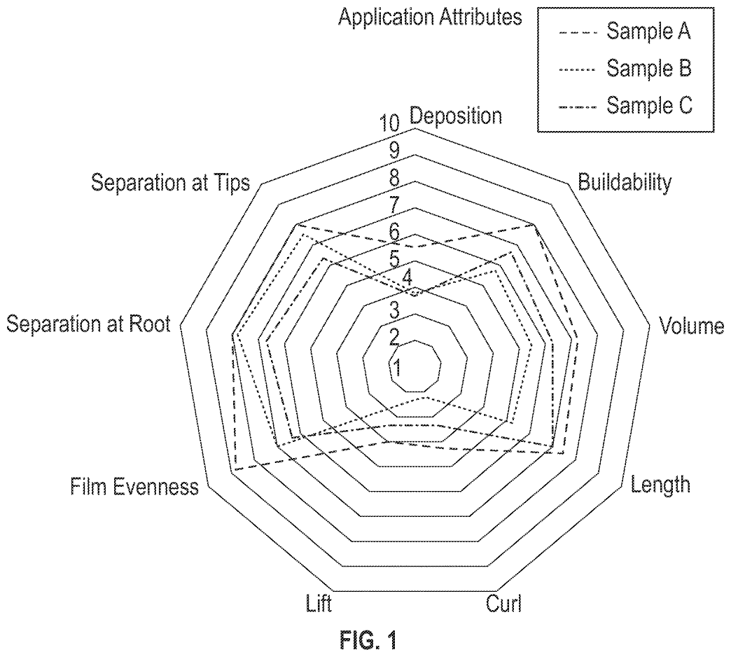

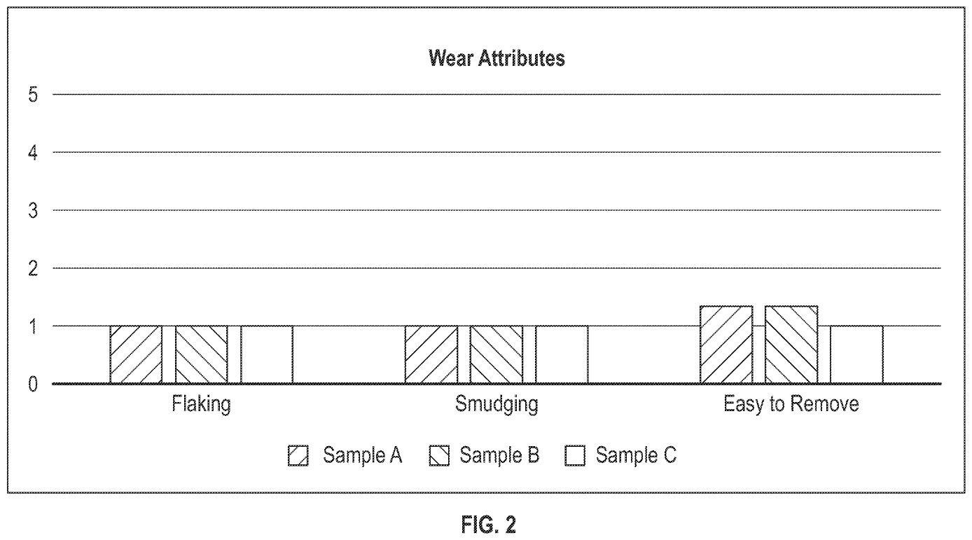

FIGS. 1 and 2 depict charts of wear data from the Examples discussed herein, gathered from panel testing as described with reference to Example 1. The Sample A with the Tara Gum and the Lipocire had beneficial wear, including reducing clumping, smudging, and flaking. Shown in FIG. 1, the Sample A had the best combination of buildability, volume, and film evenness when tested by a panel relative to the other Samples. Shown in FIG. 2, Sample A had equal or better flaking, smudging, and removal relative the other Samples.

Additionally, the Samples A to C were tested for the visual effect of the thickener in the formulation. Four panelists tested Samples A to C. The mascara formulations were applied with the same brush and in the same manner on all for panelists. Photographs were then taken of the panelists' eyes with the applied Samples. Photos were taken with the panelists facing forward and upwards, both with and without the Samples formulations applied.

The Sample A produced good coverage and volume from all angles. Sample B and Sample C also produced good coverage, however, Sample B and Sample C produced uneven coverage that was prone to clumping. Sample A produced less visible clumping. Additionally, as can be seen in the photos of panelists' eyes looking up, the root coverage was sparse in Sample C.

### Example 3 Mascara Application

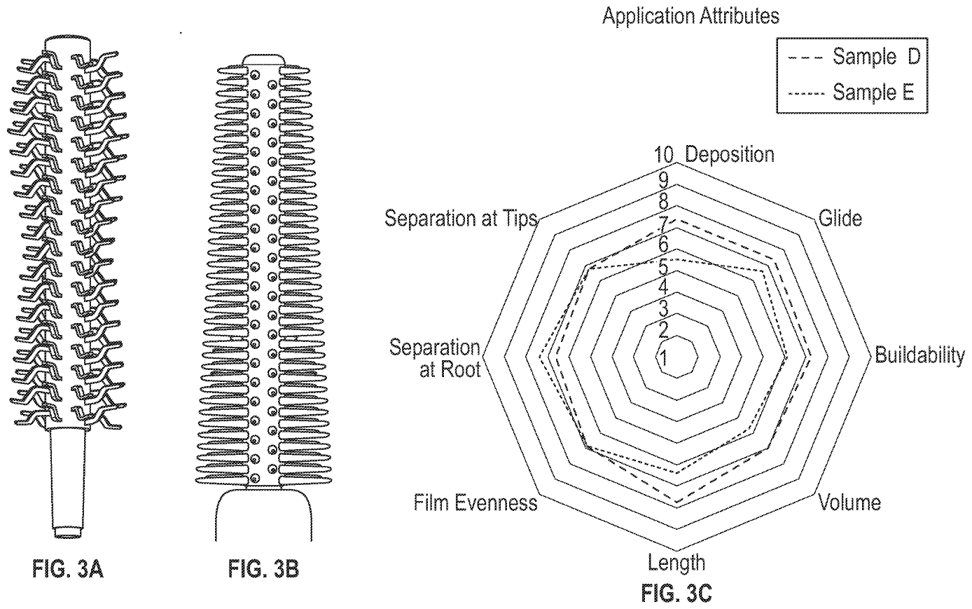

The Sample A was tested with two different mascara applicators, shown in FIG. 3A and FIG. 3B respectively. The brush from FIG. 3A was used for Sample D, while the brush from FIG. 3B was used for Sample E.

In this testing, a calibrated internal panel was used to apply and evaluate the mascaras on the panelist's lashes. Based on this, deposition, buildability, volume, and length were tested. The results of this panel testing are shown in FIG. 3C and summarized in Table 6 and Table 7:

TABLE 6
Mascara Applicator Testing.
Sample D

| | | | | | | Film | Separation | Separation |
|---|---|---|---|---|---|---|---|---|
| # | Deposition | Glide | Buildability | Volume | Length | Evenness | at Root | at Tips |
| 1 | 8 | 7 | 8 | 7 | 8 | 7 | 6 | 7 |
| 2 | 6 | 8 | 6 | 7 | 7 | 7 | 7 | 7 |
| 3 | 8 | 8 | 8 | 7 | 8 | 7 | 7 | 7 |
| 4 | 7 | 7 | 6 | 7 | 7 | 7 | 7 | 7 |
| 5 | 7 | 8 | 6 | 7 | 9 | 6 | 7 | 7 |
| 6 | 7 | 7 | 8 | 7 | 8 | 6 | 7 | 6 |
| 7 | 8 | 7 | 8 | 7 | 7 | 8 | 6 | 6 |
| 8 | 8 | 7 | 8 | 7 | 8 | 7 | 6 | 7 |
| Avg | 7.38 | 7.38 | 7.25 | 7.00 | 7.75 | 6.88 | 6.63 | 6.75 |

TABLE 6
Mascara Applicator Testing.
Sample E

| | | | | | | Film | Separation | Separation |
|---|---|---|---|---|---|---|---|---|
| # | Deposition | Glide | Buildability | Volume | Length | Evenness | at Root | at Tips |
| 1 | 5 | 6 | 6 | 5 | 6 | 8 | 8 | 7 |
| 2 | 5 | 7 | 6 | 6 | 6 | 7 | 7 | 7 |
| 3 | 5 | 7 | 7 | 6 | 7 | 7 | 8 | 7 |
| 4 | 6 | 7 | 6 | 6 | 7 | 7 | 8 | 7 |
| 5 | 6 | 7 | 5 | 6 | 7 | 6 | 7 | 6 |
| 6 | 6 | 6 | 6 | 6 | 6 | 6 | 6 | 6 |
| 7 | 6 | 6 | 7 | 5 | 7 | 7 | 7 | 7 |
| 8 | 5 | 7 | 6 | 6 | 5 | 7 | 8 | 7 |
| Avg | 5.5 | 6.63 | 6.13 | 5.75 | 6.38 | 6.88 | 7.38 | 6.75 |

Overall, the Sample D brush with the formulation of Sample A performed well.

### Various Aspects

The following exemplary aspects are provided. Each of these non-limiting aspects can stand on its own, or can be combined in various permutations or combinations with one or more of the other examples.

In some aspects, the techniques described herein relate to a cosmetic formulation for mascara, the formulation including: an emulsifier; a water phase including a thickener including a 0.3% to 1.0% Tara gum sufficiently dispersed in the water phase, wherein the water phase includes a viscosity of at least 25,000 CPS on a Brookfield measurement; and an oil phase including a wax system being about 5% to about 25% of the formulation, the wax system including a first wax having a melting point below 50° C. and a second wax having a melting point above 60° C.

In some aspects, the techniques described herein relate to a formulation, wherein the emulsifier includes sucrose stearate.

In some aspects, the techniques described herein relate to a formulation, wherein the emulsifier includes sucrose stearate and glyceryl stearate at a ratio of 1:1.

In some aspects, the techniques described herein relate to a formulation, wherein the emulsifier in 0.1% to 1% of the formulation.

In some aspects, the techniques described herein relate to a formulation, wherein the water phase further includes a film forming system of acrylates copolymer and VP hexadecane copolymer.

In some aspects, the techniques described herein relate to a formulation, wherein the acrylates copolymer and the VP hexadecane are about 1.0 wt % to about 20.0 wt % of the formulation.

In some aspects, the techniques described herein relate to a formulation, wherein the acrylates copolymer and VP hexadecane copolymer are supplied at a ratio of about 10:1 to about 20:1.

In some aspects, the techniques described herein relate to a formulation, wherein the first wax includes C10-18 triglycerides, shea butter, berry wax, myrica, tribehenin, or combinations thereof.

In some aspects, the techniques described herein relate to a formulation, wherein the first wax has a melting point of about 20 to about 50° C.

In some aspects, the techniques described herein relate to a formulation, wherein the first wax has a penetration value of about 0 to 30.

In some aspects, the techniques described herein relate to a formulation, wherein the first wax includes about 10 to about 50% of the wax system.

In some aspects, the techniques described herein relate to a formulation, wherein the second wax includes carnauba wax, euphorbia cerifera cera, cera microcristallina, hydrogenated vegetable oil, stearyl stearate, stearic acid, and combinations thereof.

In some aspects, the techniques described herein relate to a formulation, wherein the second wax has a melting point of about 60 to about 90° C.

In some aspects, the techniques described herein relate to a formulation, wherein the second wax has a penetration value of about 0 to 10.

In some aspects, the techniques described herein relate to a formulation, wherein the formulation has a viscosity of about 3 million to 6 million when measured

In some aspects, the techniques described herein relate to a formulation, wherein the formulation has a yield stress of about 300 Pa to about 2,000 Pa.

In some aspects, the techniques described herein relate to a formulation, wherein the formulation film is not compromised after rub testing at 37 C with 60 relative humidity after 15 minutes.

In some aspects, the techniques described herein relate to a formulation, wherein the formulation does not transfer from a substrate until at least 500 abrasion passes.

In some aspects, the techniques described herein relate to a formulation, further including one or more chelating agents, defoaming agents, pigments, plasticizers, or combinations thereof.

In some aspects, the techniques described herein relate to a method 1, the method including: mixing components of the water phase; mixing the components of the oil phase; and mixing the water phase and the oil phase to form the oil in water emulsion.

Additional example aspects are discussed below. The following aspects are provided, the numbering of which is not to be construed as designating levels of importance:
Aspect 1 provides a mascara formulation that is an oil in water emulsion comprising: a water phase comprising: about 0.3 wt % to about 0.7 wt % of sufficiently dispersed Tara gum (CAS No. 39300-88-4) to yield a water phase exhibiting a Brookfield viscosity of at least 0.50 CPS; an oil phase comprising: an emulsifier selected from emulsifiers classified as natural origin cosmetic ingredients (NOI) in a range of from about 0.1 to about 1 as determined by ISO 16128-1:2016 and the emulsifier does not comprise triethanolamine; a wax system in a range of from about 5 wt % to about 25 wt % of the composition wherein, when applied, the mascara formulation exhibits sufficient volume and sufficient definition as assessed by Example 1, Example 2, or both.
Aspect 2 provides the mascara formulation of Aspect 1, wherein the water phase comprises 0.4 wt % to 0.6 wt % of sufficiently dispersed Tara gum.
Aspect 3 provides the mascara formulation of any one of Aspects 1 or 2, wherein the Tara gum is considered sufficiently dispersed if the mascara formulation is free of visual agglomerations.
Aspect 4 provides the mascara formulation of any one of Aspects 1-3, wherein the wax system is in a range of from about 10 wt % to about 20 wt % of the composition.
Aspect 5 provides the mascara formulation of any one of Aspects 1-4, wherein the mascara formulation is applied with an elastomeric brush, twisted-wire brush, or both.
Aspect 6 provides the mascara formulation of any one of Aspects 1-5, wherein the wax system comprises a first wax having a melting point in a range of from about 66° C. to about 75° C., a second wax having a melting point in a range of from about 46° C. to about 56° C., a third wax having a melting point in a range of from about 78° C. to about 88° C., a fourth wax having a melting point in a range of from about 43° C. to about 57° C., or a mixture thereof.
Aspect 7 provides the mascara formulation of any one of Aspects 1-6, wherein the wax system comprises a first wax having a melting point in a range of from about 68° C. to about 73° C., a second wax having a melting point in a range of from about 48° C. to about 54° C., a third wax having a melting point in a range of from about 80° C. to about 86° C., a fourth wax having a melting point in a range of from about 45° C. to about 55° C., or a mixture thereof.
Aspect 8 provides the mascara formulation of any one of Aspects 6 or 7, wherein a ratio between the first wax, the second wax, the third wax, and the fourth wax is in a range of from about 1:1:0.3:0.8 to about 1:1:0.5:0.9.
Aspect 9 provides the mascara formulation of any one of Aspects 6-8, wherein a ratio between the first wax, the second wax, the third wax, and the fourth wax is 1:1:0.4:0.8.
Aspect 10 provides the mascara formulation of any one of Aspects 6-9, wherein the first wax is carnauba wax, the second wax is myrica wax, the third wax is berry wax, and the fourth wax is candelilla wax.
Aspect 11 provides the mascara formulation of any one of Aspects 1-10, wherein the mascara formulation is free of xantham, hydroxyethyl cellulose, acacia gum, or a mixture thereof.
Aspect 12 provides the mascara formulation of any one of Aspects 1-11, wherein the mascara formulation is a homogenized composition.
Aspect 13 provides the mascara formulation of any one of Aspects 1-12, wherein the emulsifier includes sucrose stearate.
Aspect 14 provides the mascara formulation of any one of Aspects 1-13, wherein the wax system comprises one or more synthetically prepared waxes.
Aspect 15 provides the mascara formulation of any one of Aspects 1-14, wherein application comprises applying no more than five strokes of the mascara formulation to a keratinous tissue.
Aspect 16 provides the mascara formulation of any one of Aspects 1-15, wherein the emulsifier is in a range of from about 1 wt % to about 15 wt % of the mascara formulation.
Aspect 17 provides the mascara formulation of any one of Aspects 1-16, wherein the emulsifier is in a range of from about 1 wt % to about 5 wt % of the mascara formulation.
Aspect 18 provides a method of using a mascara formulation, the method comprising: contacting a first portion of the mascara formulation with a keratinous tissue with a stroke from a brush; optionally contacting a second portion of the mascara formulation with the keratinous tissue with a stroke from the brush, wherein the mascara formulation is an oil in water emulsion comprising: a water phase comprising: about 0.3 wt % to about 0.7 wt % of sufficiently dispersed Tara gum (CAS No. 39300-88-4) to yield a water phase exhibiting a Brookfield viscosity of at least 0.50 CPS; an oil phase comprising: an emulsifier selected from emulsifiers classified as natural origin cosmetic ingredients (NOI) having a minimum value of 1 as determined by ISO 16128-1:2016 and the emulsifier does not comprise triethanolamine; a wax system in a range of from about 5 wt % to about 25 wt % of the composition, wherein, when applied, the mascara formulation exhibits sufficient volume and sufficient definition as assessed by Example 1.
Aspect 19 provides the method of Aspect 18, wherein the water phase comprises 0.4 wt % to 0.6 wt % of sufficiently dispersed Tara gum.
Aspect 20 provides the method of any one of Aspects 18 or 19, wherein the Tara gum is considered sufficiently dispersed if the mascara formulation is free of visual agglomerations.
Aspect 21 provides the method of any one of Aspects 18-20, wherein the wax system is in a range of from about 10 wt % to about 20 wt % of the composition.
Aspect 22 provides the method of any one of Aspects 18-21, wherein the mascara formulation is applied with an elastomeric brush, twisted-wire brush, or both.
Aspect 23 provides the method of any one of Aspects 18-22, wherein the wax system comprises a first wax having a melting point in a range of from about 66° C. to about 75° C., a second wax having a melting point in a range of from about 46° C. to about 56° C., a third wax having a melting point in a range of from about 78° C. to about 88° C., a fourth wax having a melting point in a range of from about 43° C. to about 57° C., or a mixture thereof.
Aspect 24 provides the method of any one of Aspects 18-23, wherein the wax system comprises a first wax having a melting point in a range of from about 68° C. to about 73° C., a second wax having a melting point in a range of from about 48° C. to about 54° C., a third wax having a melting point in a range of from about 80° C. to about 86° C., a fourth wax having a melting point in a range of from about 45° C. to about 55° C., or a mixture thereof.
Aspect 25 provides the method of any one of Aspects 23 or 24, wherein a ratio between the first wax, the second wax, the third wax, and the fourth wax is in a range of from about 1:1:0.3:0.8 to about 1:1:0.5:0.9.
Aspect 26 provides the method of any one of Aspects 23-25, wherein a ratio between the first wax, the second wax, the third wax, and the fourth wax is 1:1:0.4:0.8.
Aspect 27 provides the method of any one of Aspects 23-26, wherein t the first wax is carnauba wax, the second wax is myrica wax, the third wax is berry wax, and the fourth wax is candelilla wax.
Aspect 28 provides the method of any one of Aspects 18-27, wherein the mascara formulation is free of xantham.
Aspect 29 provides the method of any one of Aspects 18-28, wherein the mascara formulation is a homogenized composition.
Aspect 30 provides the method of any one of Aspects 18-29, wherein the emulsifier includes sucrose stearate.
Aspect 31 provides the method of any one of Aspects 18-30, wherein the wax system comprises one or more synthetically prepared waxes.
Aspect 32 provides the method of any one of Aspects 18-31, wherein application comprises applying no more than two strokes of the mascara formulation to a keratinous tissue.
Aspect 33 provides the method of any one of Aspects 18-32, wherein the emulsifier is in a range of from about 1 wt % to about 15 wt % of the mascara formulation.
Aspect 34 provides the method of any one of Aspects 18-33, wherein the emulsifier is in a range of from about 1 wt % to about 5 wt % of the mascara formulation.
Aspect 35 provides a method of making the mascara formulation of any one of Aspects 1-34, the method comprising: mixing components of the water phase; mixing the components of the oil phase; and mixing the water phase and the oil phase to form the oil in water emulsion.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

The terms and expressions that have been employed are used as terms of description and not of limitation, and there is no intention in the use of such terms and expressions of excluding any equivalents of the features shown and described or portions thereof, but it is recognized that various modifications are possible within the scope of the embodiments of the present invention. Thus, it should be understood that although the present invention has been specifically disclosed by specific embodiments and optional features, modification and variation of the concepts herein disclosed may be resorted to by those of ordinary skill in the art, and that such modifications and variations are considered to be within the scope of embodiments of the present invention.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A cosmetic formulation for mascara, the formulation comprising:
an emulsifier;
a water phase comprising a thickener including a 0.3 wt % to 1.0 wt % Tara gum sufficiently dispersed in the water phase, wherein the water phase comprises a viscosity of at least 25,000 CPS on a Brookfield measurement; and
an oil phase comprising a wax system being about 5 wt % to about 25 wt % of the formulation, the wax system including a first wax having a melting point below 50° C. and a second wax having a melting point above 60° C.

2. The formulation of claim 1, wherein the emulsifier comprises sucrose stearate.

3. The formulation of claim 2, wherein the emulsifier comprises sucrose stearate and glyceryl stearate at a ratio of 1:1 by weight.

4. The formulation of claim 1, wherein the emulsifier comprises 0.1 wt % to 1 wt % of the formulation.

5. The formulation of claim 1, wherein the water phase further comprises a film forming system of acrylates copolymer and VP hexadecene copolymer.

6. The formulation of claim 5, wherein the acrylates copolymer and the VP hexadecene are about 1.0 wt % to about 20.0 wt % of the formulation.

7. The formulation of claim 5, wherein the acrylates copolymer and VP hexadecene copolymer are supplied at a ratio of about 10:1 by weight to about 20:1 by weight.

8. The formulation of claim 1, wherein the first wax comprises at least on of C10-18 triglycerides, shea butter, berry wax, myrica, or tribehenin.

9. The formulation of claim 1, wherein the first wax has a melting point of about 20° C. to about 50° C.

10. The formulation of claim 1, wherein the first wax has a penetration value of about 0 to 30.

11. The formulation of claim 1, wherein the first wax comprises about 10 wt % to about 50 wt % of the wax system.

12. The formulation of claim 1, wherein the second wax comprises at least one of carnauba wax, euphorbia cerifera cera, cera microcristallina, hydrogenated vegetable oil, stearyl stearate, or stearic acid.

13. The formulation of claim 1, wherein the second wax has a melting point of about 60° C. to about 90° C.

14. The formulation of claim 1, wherein the second wax has a penetration value of about 0 to 10.

15. The formulation of claim 1, wherein the formulation has a viscosity between about 3 million CPS and 6 million CPS when measured.

16. The formulation of claim 1, wherein the formulation has a yield stress of about 300 Pa to about 2,000 Pa.

17. The formulation of claim 1, wherein the formulation produces a film that is not compromised after rub testing at 37° C. with 60 relative humidity after 15 minutes.

18. The formulation of claim 1, wherein the formulation does not transfer from a substrate until at least 500 abrasion passes.

19. A method of applying the mascara formulation of claim 1, the method comprising applying the mascara formulation with a mascara brush having a plurality of bristles extending outward from a stem, each of the plurality of bristles having a bend.

20. A method of making the mascara formulation of claim 1, the method comprising:
mixing components of the water phase;
mixing the components of the oil phase; and
mixing the water phase and the oil phase to form the oil in water emulsion.
